(19) United States
(12) Patent Application Publication
(10) Pub. No.: US 2025/0124143 A1
(43) Pub. Date: Apr. 17, 2025

(54) Transferring Encrypted Data from a Medical Technology System

(71) Applicant: Siemens Healthineers AG, Forchheim (DE)

(72) Inventors: Swen Campagna, Engelthal (DE); Thomas Beck, Dormitz (DE)

(73) Assignee: Siemens Healthineers AG, Forchheim (DE)

(21) Appl. No.: 18/917,186

(22) Filed: Oct. 16, 2024

(30) Foreign Application Priority Data
Oct. 17, 2023 (DE) 10 2023 210 144.0

(51) Int. Cl. G06F 21/60; G16H 10/60; G16H 80/00

(52) U.S. Cl. CPC G06F 21/602; G16H 10/60; G16H 80/00

(57) ABSTRACT

Method for transmitting encrypted data from a medical technology system, including: generating the encrypted data by applying a DK to data generated by the MTS; transmitting a first user-specific DK for decrypting the encrypted data to a first data user authorized by a central data transfer authorization entity; transmitting the encrypted data from the MTS to the first data user; decrypting the encrypted data by the first data user by applying the first user-specific DK; transmitting a second user-specific DK, which is different from the first user-specific DK, by the central data transfer authorization entity to a second data user authorized by the central data transfer authorization entity for decrypting the encrypted data; transmitting the encrypted data by the MTS or by the first data user to the second data user; and decrypting the encrypted data by the second data user by applying the second user-specific DK.

### TECHNICAL FIELD

The disclosure relates to a method for transmitting encrypted data from a medical technology system. Furthermore, the disclosure relates to a data transfer system.

### BACKGROUND

During the operation of medical technology systems, in particular during the operation of imaging medical technology systems, such as, for example, magnet resonance imaging systems (abbreviated to MR systems) or computed tomography systems (abbreviated to CT systems), data, in particular so-called machine data, is produced in predominantly digital form. This type of data primarily comprises transmission and reception data, control data, sensor data for system monitoring, etc. In addition, log data from software processes, also known as logging information, is generated and stored persistently. Such logging information can, for example, comprise an error number, a warning message, an error message, or an information message.

In addition, many digital processes are recorded in detail in so-called log files (also referred to as “logging”), both during the course of an examination and during an individual measurement, in order, in the event of an error, to be able to identify the cause of the error as quickly and specifically as possible and to enable the medical technology system to be improved.

Access to this information allows a very precise understanding of the exact function of a medical technology system and its subcomponents. In addition, this access allows the understanding of existing implicit functionalities and intermediate results that the user does not need to know in order to work with a medical technology system but which have a major influence on the quality of the examination and, thus, the performance of the overall system. In the case of a magnetic resonance imaging system, this may entail, for example, patient-specific adjustments, tune-up measurements, SAR calculations, sensor data from temperature sensors, etc. Such data are internal intermediate results that are available as both raw data and internal result data and are generally stored in the aforementioned logging files, which are not part of the actual images generated but are very relevant for their high-quality production.

Hereinafter, the aforementioned internal data from a medical technology system, which does not directly concern medical content but relates to technical aspects of the medical technology system, will be referred to as machine data.

However, it may be of great interest for manufacturers to control or at least monitor the further dissemination of the aforementioned data to third parties in order to avoid having unnecessarily disclose knowledge of internal technical processes of their own medical technology systems when in competition with competitors.

There is, therefore, a problem with monitoring or even controlling the transfer of data from medical technology systems, in particular machine data that is generated and stored during operation, to third parties.

### SUMMARY

This object is achieved by a method for transmitting encrypted data from a medical technology system and a data transfer system.

In the method according to the disclosure for transmitting encrypted data from a medical technology system, preferably a magnetic resonance system or a computed tomography system, the encrypted data is generated by applying a data key or encryption key, preferably a data key specific to the medical technology system, to data, preferably to machine data, generated by the medical technology system. As will be explained in detail later, the data which is generated by the medical technology system and then stored in encrypted form, preferably on a computer system of the medical technology system, can have a very different content, but the application is intended to focus in particular on the aforementioned machine data, which relates to or comprises valuable and sensitive information about internal technical processes of the medical technology system. Machine data should comprise all data that describes and characterizes the technical processes running in a machine, in particular, a medical technology system. Machine data should, in particular, comprise data from a medical technology system in the aforementioned log files. As already mentioned, in the case of a magnetic resonance system, the log files comprise patient-specific adjustments, tune-up measurements, and SAR calculations. Further internal machine data relates to monitoring gradient activity for stimulation limitation, information about the position of the patient bench, and temperature values from temperature sensors, for example, from cooling circuits. In the case of CT systems, the machine data comprises the applied X-ray dose, internal error messages, information relating to the energy consumption of individual components, and status information relating to data transmission, in particular via a slip ring.

It should be emphasized at this point that, in addition to the aforementioned machine data, other data that occurs during the operation of a medical technology system, in particular measurement data from an examination object and image data from such an examination object, can also be stored in encrypted form on a computer system assigned to the medical technology system.

The encrypted data is now stored on the computer system of the medical technology system and can be decrypted and read or processed using the specific data key or decryption key of the medical technology system by authorized people or facilities, for example radiologists.

If an external person or external data processing facility, hereinafter referred to as the first data user, preferably a maintenance person or a computer system assigned to a maintenance person or maintenance facility, now wishes to access the encrypted data of the medical technology system, the person or computer system in question must not simply have arbitrary access to the internal data key of the medical technology system, instead, with the method according to the disclosure, it is first necessary to contact a central data transfer authorization entity. If the first data user is authorized by the central data transfer authorization entity to access the encrypted data of the medical technology system, a first user-specific data key for decrypting the encrypted data of the medical technology system is transmitted by the central data transfer authorization entity to the first data user authorized by the central data transfer authorization entity.

As will be explained in detail later, it may be the case that, after authorization, the computer system of the medical technology system receives a data key matching the first user-specific data key as an encryption key from the central data transfer authorization entity and uses this matching data key or encryption key to generate an encrypted version of the data stored on the computer system. It can also be the case that the first user-specific data key or decryption key is coordinated with the data key or encryption key primarily used by the computer system, and therefore, the first user-specific data key or decryption key can be used to access the primarily encrypted data.

Once the first data user has received the first user-specific data key or decryption key, said user requests the encrypted data from the computer system of the medical technology system. The encrypted data is then transmitted to the first data user by the medical technology system or by the computer system assigned to this medical technology system.

When the first data user has received the encrypted data, the encrypted data is decrypted by the first data user by applying the first user-specific data key.

If a second data user, preferably a second maintenance facility or maintenance person, also wishes to access the encrypted data of the medical technology system and is trusted by the central data transfer authorization entity, a second user-specific data key, which is different from the first user-specific data key, is transmitted from the central data transfer authorization entity to the second data user authorized by the central data transfer authorization entity in order to decrypt the encrypted data.

Subsequently, the second data user can request the encrypted data of the medical technology system from either the actual computer system of the medical technology system or from the first data user, and the encrypted data is then transmitted from the medical technology system or from the first data user to the second data user.

Finally, the encrypted data is decrypted by the second data user by applying the second user-specific data key.

Similarly, any number of second data users can participate in the transmission process, wherein each of the second data users uses a specific key to decrypt the encrypted data. Furthermore, it is also possible for a plurality of medical technology systems to participate in the data transmission. Since the respective medical technology systems use different keys to encrypt the data to be transmitted by them, the central data transfer authorization entity can even control and at least monitor which user receives which data from which medical system by assigning system-specific keys or decryption keys to specific data users. This enables very detailed monitoring and control of data transfer between individual participants in a data transfer system.

Of course, computer programs must be installed in data users' computing systems to prevent the transmission of unencrypted data. This prevents received data from being forwarded after decryption to any potentially unauthorized user who does not have a suitable data key.

The central data transfer authorization entity, in particular a manufacturer, often has an interest in ensuring that encrypted data, in particular, encrypted machine data, of a medical technology system manufactured or managed by the central data transfer authorization entity is not forwarded to third parties in an uncontrolled manner. Even if the central data transfer authorization entity cannot prevent machine data from being forwarded to third parties for legal reasons, the data transfer authorization entity at least becomes aware of the fact that it has been forwarded and of the recipient, and the data is not forwarded indiscriminately to any recipients.

The data transfer system, according to the disclosure, has a medical technology system that is configured to generate encrypted data generated by a medical technology system by applying a data key, which is preferably specific to the medical technology system, to this data of the medical technology system. A medical technology system comprises technical means for the medical treatment of a patient. Such medical treatment can comprise an examination, an evaluation of an examination, or a therapeutic procedure. In particular, magnetic resonance imaging systems and computed tomography systems are covered by the term “medical technology system.”

The data transfer system, according to the disclosure, also has a first data user. Such a first data user can, for example, comprise a facility that is concerned with the maintenance of the medical technology system or a facility that is concerned with the medical treatment of a patient and, therefore, requires examination data from the medical technology system, preferably image data.

The data transfer system, according to the disclosure, has a central data transfer authorization entity that is configured to authorize the first data user to receive the encrypted data from the medical technology system and to transmit the first user-specific data key for decrypting the encrypted data to the first data user authorized by the central data transfer authorization entity.

The medical technology system is configured to transmit the encrypted data to the first data user.

The first data user is configured to decrypt the encrypted data by applying the first user-specific data key.

The data transfer system, according to the disclosure, also comprises a second data user. The second data user can be an alternative maintenance facility, which, instead of the maintenance facility possibly assigned to the first data user, manages or performs maintenance processes on the medical technology system.

The central data transfer authorization entity is configured to authorize the second data user and to transmit a second user-specific data key or decryption key, which is different from the first user-specific data key, for decrypting the encrypted data to the second data user authorized by the central data transfer authorization entity to receive the encrypted data.

The medical technology system and/or the first data user are configured to transmit the encrypted data to the second data user.

The second data user is configured to decrypt the encrypted data by applying the second user-specific data key or decryption key.

It should be mentioned at this point that the data transfer system, according to the disclosure, can have any number of second data users participating in the transmission process, wherein each of the second data users uses a specific key for decrypting the encrypted data. Furthermore, a plurality of medical technology systems can also participate in the data transmission. Since the respective medical technology systems use different keys to encrypt the data transmitted by them, the central data transfer authorization entity can even control and monitor which user receives data from which medical technology system by assigning system-specific keys to specific users. This makes it possible to monitor and control the forwarding of data.

The data transfer system, according to the disclosure, shares the advantages of the method according to the disclosure for the monitored transmission of encrypted data from a medical technology system.

A large part of the aforementioned components of the data transfer system, according to the disclosure, can be implemented in whole or in part in the form of software modules in a processor of a corresponding computer system, for example, by a computer system in the central data transfer authorization entity or a computer system assigned to the medical technology system or a computer system assigned to one of the data users. A largely software-based implementation has the advantage that previously used computing systems can be easily retrofitted by means of a software update in order to operate in the manner according to the disclosure. In this respect, the object is also achieved by a corresponding computer program product with a computer program that can be loaded directly into one or more computing systems with program sections for executing the steps of the method according to the disclosure for transmitting encrypted data from a medical technology system when the program is executed in the computing system or systems. In addition to the computer program, such a computer program product can optionally comprise additional items, such as, for example, documentation and/or additional components, including hardware components, such as, for example, hardware keys (dongles, etc.) for using the software.

A computer-readable medium, for example, a memory stick, a hard disk, or another kind of transportable or permanently installed data carrier on which the program sections of the computer program that can be read in and executed by a computing system are stored can be used for transport to the computing system and/or for storage on or in the computing system. For this purpose, the computing system can, for example, have one or more interacting microprocessors or the like.

The dependent claims and the following description in each case contain particularly advantageous aspects and developments of the disclosure. Herein, in particular, the claims of one claim category can also be developed analogously to the dependent claims of another claim category. In addition, it is also possible in the context of the disclosure for the different features of different exemplary aspects and claims to be combined to form new, exemplary aspects.

As already mentioned, in one variant of the method, according to the disclosure for transmitting encrypted data from a medical technology system, the second user-specific data key has a plurality of different data keys. In this variant, the second data user has a plurality of different data users and a different second user-specific data key, which is different from the first user-specific data key and the other second user-specific data keys, is transmitted to each of the different second data users by the central data transfer authorization entity.

The encrypted data is transmitted to the respective second data user upon request by the medical technology system or by the first data user.

Once the encrypted data has been received, it is decrypted by the respective second data user by applying the respective second user-specific data key.

Advantageously, access by a plurality of data users to the data generated and encrypted by the medical technology system is monitored and possibly controlled. Advantageously, a user of the medical technology system can assign a plurality of different tasks for the maintenance of the medical technology system or for the evaluation of data from the medical technology system to a plurality of different service providers without the data being disseminated in an uncontrolled manner, since a central, possibly authorizing, monitoring entity is in each case involved in the transfer of the data.

As already mentioned, the data generated by the medical technology system preferably comprises data of one of the following data types:
machine data,
measurement data from a region of interest of a patient,
medical image data from a region of interest.

Machine data primarily comprises transmission and reception data, control data, and internal measurement data, in particular, sensor data for system monitoring, etc.

If the data comprises machine data, the machine data preferably comprises technical log data.

The log data comprises data from software processes, also known as logging information, which is stored persistently, preferably in log files. Such logging information can, for example, comprise a warning message, an error message, or an information message.

In addition, many digital processes are recorded in detail in log files of this type for storing logging information, both during the course of an examination and during an individual measurement, in order, in the event of an error, to be able to identify the cause of the error as quickly and specifically as possible and to enable the medical technology system to be improved.

As already mentioned in the introduction, access to this information allows a very precise understanding of the exact function, time sequences, and implementation details of a medical technology system and its subcomponents. In addition, this access allows an understanding of existing implicit functionalities and intermediate results that the user does not need to know in order to work with a medical technology system but which have a great influence on the quality of the examination and, thus, the performance of the overall system. In the case of a magnetic resonance imaging system, this may entail, for example, patient-specific adjustments, tune-up measurements, SAR calculations, etc. Such data are internal intermediate results that are available as both raw data and internal result data and are generally stored in the aforementioned logging files, which are not part of the actual images generated but are very relevant for their high-quality production.

In one aspect of the method for transmitting encrypted data from a medical technology system, the first user-specific data key comprises a first key pair with a first private data key and a first public data key. Therefore, this variant uses the public/private key principle to encrypt and decrypt data that is to be transferred.

Furthermore, the second user-specific data key comprises a second key pair with a second private data key or decryption key and a second public data key or encryption key.

The first public data key is transmitted by the first data user to the medical technology system.

The encrypted data is decrypted by the computer system of the medical technology system and re-encrypted with the first public data key or encryption key before being transmitted to the first data user and decrypted by the first data user after receipt with the first private data key or decryption key.

In addition, the second public data key is transmitted by the second data user to the medical technology system or to the first data user.

The encrypted data is decrypted by the computer system of the medical technology system and encrypted with the second public data key or encryption key before being transmitted to the second data user and decrypted by the second data user after receipt with the second private data key or decryption key.

Advantageously, if required, a data user can itself transmit a public data key to a data source, in particular, of a computer system of a medical technology system, and thus request encrypted data directly from this data source with this public key. Nevertheless, the central data transfer authorization entity retains control over access to the encrypted data since the respective data user must first request a key pair with a private and a public data key from the central data transfer authorization entity. However, in this variant, data users have a certain amount of freedom to partially circumvent monitoring by forwarding or exchanging key pairs. Of course, computer programs must be installed in the data users' computing systems to prevent the transmission of unencrypted data.

In one variant of the method, according to the disclosure for transmitting encrypted data from a medical technology system, the first and/or second user-specific data key has a limited validity period. Advantageously, an access period is limited in order to obtain data from the medical technology system. Advantageously, a user cannot have authorization to access the data from a medical technology system ‘in stock’ if this is not desired.

Alternatively, the user-specific data keys can also be assigned a specific authorization level. Advantageously, a data user's access can be restricted to a subset of the data generated by the medical technology system data, in particular machine data.

In an advantageous aspect of the method according to the disclosure for transmitting encrypted data from a medical technology system, a pool of public data keys is transmitted to the medical technology system by the central data transfer authorization entity.

The encrypted data is first decrypted by the computer system of the medical technology system and encrypted with one of the pool's public keys before being transmitted to the first data user.

After receiving the encrypted data, the first data user requests a first private data key from the central data transfer authorization entity. In this variant, the encryption and transfer of encrypted data from the medical technology system to one of the data users can also take place without prior authorization by the central data transfer authorization entity. This is because a public data key and an associated private data key are distributed separately to the sender and recipient and the sender has public data keys so-to-speak ‘in stock’.

Finally, the encrypted data transmitted to the first data user is decrypted with the first private data key. In this variant, although data is encrypted and correspondingly exported or transferred by a sender without further approval from the central data transfer authorization entity, the central data transfer authorization entity can use the distribution or assignment of the private data key to influence who can decrypt which data from whom, and thus also process said data.

In one variant of the method, according to the disclosure for transmitting encrypted data from a medical technology system, the central data transfer authorization entity transmits a pool of public second data keys to the first data user. In this variant, the first data user is enabled to automatically transmit encrypted data to other data users, in particular a second data user, using the public second data keys received.

The first data user encrypts the data to be transmitted with one of the public second data keys received and transmits the data encrypted in this way to the second data user.

Once the encrypted data has been received, the second data user requests the second private key assigned to the public second data key used by the first data user from the central data transfer authorization entity and, after receipt of the second private data key, decrypts the encrypted data with the second private data key. Therefore, the central data transfer authorization entity can also monitor the data transfer between the first data user and a second data user by controlling who can send encrypted data and who can decrypt this data by assigning the pool of public data keys to the first user and a matching private data key to the second data user. Of course, computer programs must be installed in the data users' computing systems to prevent the transmission of unencrypted data. Alternatively, the encrypted data can also only be accessed with the aid of a data decryption program and with knowledge of the matching data key. In this case, the decrypted data is never located on the system of the first or second data user, but is only decrypted ‘on-the-fly’. This implementation is also possible for all variants and is definitely advantageous. This is because it prevents the protection mechanisms from being circumvented by the first or second user. The procedure can even be applied directly to the medical technology system.

In one aspect of the method, according to the disclosure for monitored transmission of encrypted data from a medical technology system, the first user-specific data key comprises a user- and system-specific data key for decrypting the data encrypted by applying a data key specific to the medical technology system and the second user-specific data key also comprises a user- and system-specific data key for decrypting the data encrypted by applying a data key specific to the medical technology system. Advantageously, an assignment between a respective medical technology system and a data user can be controlled or monitored. In particular, it is possible to prevent data from being freely transferred by forwarding data keys without the agreement of the central data transfer authorization entity.

In a specific variant of the method according to the disclosure for transmitting encrypted data from a medical technology system, the respective user-specific data key, and preferably the user- and system-specific data key, is generated on the basis of a MAC address of the medical technology system and/or a computer system assigned to the medical technology system and a MAC address of the respective data user.

If the first user- and system-specific data key is generated on the basis of a MAC address of a computer unit of the first data user and on the basis of the MAC address of the computer unit of the medical technology system, the second user- and system-specific data key is likewise generated on the basis of a MAC address of a computer unit of the second data user and on the basis of the MAC address of the computer unit of the medical technology system. Advantageously, the specificity for the data user and the specificity for the relevant medical technology system can be achieved by the fact that the data key assigned to the data user and the medical technology system only functions for data transmission between this individual data user and the individual medical technology system. Therefore, this also achieves particularly effective and detailed monitoring and control of the data exchange between a plurality of medical technology systems and data users.

In a special aspect of the method, according to the disclosure for monitored transmission of encrypted data from a medical technology system, the data key specific to the medical technology system data key is generated on the basis of a secret code generated by the data transfer authorization entity.

In this variant, the first user- and system-specific data key is generated on the basis of the secret code generated by the data transfer authorization entity, and the second user- and system-specific data key is generated on the basis of the secret code generated by the central data transfer authorization entity. In this variant, the data transmission is additionally secured such that encryption and decryption can only take place with the aid of a secret code controlled by the central data transfer authorization entity. If the encryption and decryption are additionally based on user- and system-specific data, in particular the aforementioned individual MAC addresses, the encryption can additionally take place on a user- and system-specific basis.

In a particularly preferred variant of the method, according to the disclosure for the monitored transmission of encrypted data from a medical technology system, the first user- and system-specific data key is generated on the basis of a checksum for the secret code generated by the data transfer authorization entity, the MAC address of the computer unit of the medical technology system and the MAC address of the computer unit of the first data user and the second user- and system-specific data key is generated on the basis of a checksum for the secret code generated by the data transfer authorization entity, the MAC address of the computer unit of the medical technology system and the MAC address of the computer unit of the second data user. By ascertaining the checksum, it is checked whether the release key defined by the secret code is applied to the correct or intended constellation of a medical technology system and a data user. The advantage of this is that none of the data users has a “free pass” for all possible medical technology systems and, conversely, that the respective medical technology systems cannot distribute data widely across any data users without being authorized to do so.

### BRIEF DESCRIPTION OF THE DRAWINGS

The aspects of the disclosure are explained again in more detail below with reference to the appended figures and with reference to exemplary aspects. In the figures:

FIG. 1 shows a schematic depiction of a data transfer system according to an exemplary aspect of the disclosure,

FIG. 2 shows a schematic depiction of a data transfer system according to an alternative exemplary aspect of the disclosure,

FIG. 3 shows a schematic depiction of a data transfer system according to yet another alternative exemplary aspect of the disclosure,

FIG. 4 shows a flowchart illustrating a method for the monitored transmission of encrypted data from a medical technology system according to an exemplary aspect of the disclosure,

FIG. 5 shows a flowchart illustrating a method for the monitored transmission of encrypted data from a medical technology system according to an alternative exemplary aspect of the disclosure,

FIG. 6 shows a flowchart illustrating a method for the monitored transmission of encrypted data from a medical technology system according to yet another alternative exemplary aspect of the disclosure.

### DETAILED DESCRIPTION

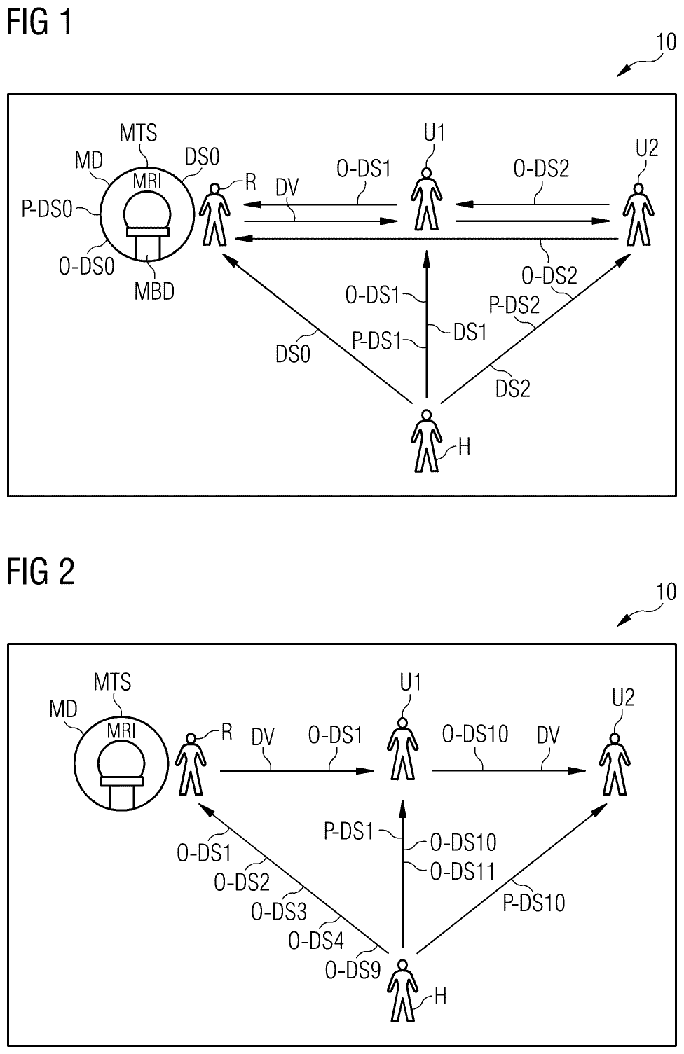

FIG. 1 is a schematic depiction of a data transfer system 10 according to an exemplary aspect of the disclosure. The data transfer system 10 has a medical technology system, MTS, for example, a magnetic resonance system with which medical image data MBD of a patient is recorded. The medical image data MBD is first appraised by a radiologist R. Furthermore, during the image recording process, in addition to the medical image data MBD, machine data MD describing the technical state of the medical technology system MTS is also generated by the medical technology system MTS.

The data transfer system 10 also comprises a manufacturer H of the medical technology system MTS as a central data transfer authorization facility which authorizes and controls the data traffic in the data transfer system 10. The manufacturer H is in possession of a plurality of key pairs DS0, DS1, DS2 with which medical image data MBD to be transferred or which has been transferred can be encrypted and decrypted. The manufacturer H first transmits a key pair DS0 assigned to the medical technology system MTS to the medical technology system MTS. The public data key O-DS0 of the key pair DS0 assigned to the medical technology system MTS is now used to encrypt the medical image data MBD and the machine data MD by a data processing facility (not shown) of the medical technology system MTS and to store them in a storage facility (not shown) in the radiology department. If the radiologist R wishes to examine the medical image data MBD, said radiologist decrypts the encrypted medical image data MBD with the private data key P-DS0 of the key pair DS0 assigned to the medical technology system and can now access the decrypted medical image data MBD.

If a first user U1, working, for example, as a physician in a special medical department in the hospital in which the medical technology system MTS is located or as a maintenance technician in this hospital, wishes to, said first user must first request a first key pair DS1 from the manufacturer H. The manufacturer H now learns from this request that the first user U1 wishes to access either the medical image data MBD or the machine data MD of the medical technology system MTS. The manufacturer H now authorizes the first user U1 to access the desired data, for example, the machine data MD of the medical technology system MTS. For this purpose, the manufacturer H transmits a first key pair DS1 to the first user U1. The first user U1 now transmits the public key O-DS1 of the first key pair DS1 to the data processing facility of the medical technology system MTS. The desired machine data MD is first decrypted by the data processing facility of the medical technology system MTS with its own private data key P-DS0 of the key pair DS0 assigned to the medical technology system MTS and re-encrypts it with the public data key O-DS1 of the first key pair DS1 and transmits it to the first user U1 in an encrypted state. The first user U1 now receives the encrypted data DV, in this case machine data MD, and decrypts it using the private data key P-DS1 of the first key pair DS1. The first user U1 can then read the machine data MD and carry out maintenance on the medical technology system MTS on the basis of the machine data MD.

If a second data user U2 now likewise wishes to access the machine data MD of the medical technology system MTS in order, for example, likewise to carry out maintenance tasks, the second data user U2 requests a second data key DS2 from the manufacturer H, which has a key pair with a public data key O-DS2 and a private data key P-DS2. The second user U2 then sends the public data key O-DS2 to either the medical technology system MTS or the first user U1. In response, either the data processing facility of the medical technology system MTS or the first user U1 encrypts the machine data MD with the second public data key O-DS2 and transmits the machine data MD encrypted in this way to the second user U2. The second user U2 can now likewise carry out maintenance work on the medical technology system MTS on the basis of the decrypted machine data MD. Therefore, in the exemplary aspect illustrated in FIG. 1, a target person for the transfer must be known and authorized at the time of the data transfer since otherwise, the data to be transmitted cannot be encrypted or decrypted. As already explained, the medical technology system MTS and the users U1, U2 are assigned computing systems which encrypt and decrypt data and only transmit encrypted data DV to other participants in the data transfer network 10 so that it is ensured that no unencrypted data, in particular unencrypted machine data MD, is transferred.

FIG. 2 is a schematic depiction of a data transfer system 10 according to an alternative exemplary aspect of the disclosure. The concept of the second exemplary aspect is that it is possible for a participant, or a subunit of the data transfer system 10 assigned to this participant, to transmit encrypted data DV to the respective recipient, even without prior authorization of the recipient by the manufacturer H. Herein, however, the recipient should only be able to read the encrypted data DV when authorized by the manufacturer H. For this purpose, the manufacturer H distributes a defined number of different public data keys O-DS1, O-DS2, . . . . O-DS11 to individual participants in the data transfer system 10. For example, the manufacturer H transmits a first group of public data keys O-DS1, O-DS2, O-DS3, O-DS4, . . . , O-DS9 to the data processing system of the medical technology system MTS. This first group of public data keys O-DS1, O-DS2, O-DS3, O-DS4, . . . , O-DS9 can now be used by the data processing facility of the medical technology system MTS as a stock of keys in order to transmit encrypted data of the medical technology system MTS to other participants in the data transfer system 10 under its own control. Likewise, the manufacturer H transmits a second group of public data keys O-DS10, O-DS11 to the first user U1 so that the first user U1 has a small stock of public data keys with which data to be transferred can be encrypted if required.

FIG. 2 symbolizes that the data processing facility of the medical technology system MTS encrypts machine data MD with a first public data key O-DS1 of the first group of public data keys O-DS1, O-DS2, O-DS3, O-DS4, . . . , O-DS9. This encrypted machine data MD is now transmitted by the data processing facility of the medical technology system MTS to the first data user U1. However, the first data user U1 cannot yet read the received encrypted machine data MD because it is encrypted with the first public data key O-DS1, and the user U1 does not yet have the first public data key O-DS1 that matches the first private data key P-DS1. Once the encrypted machine data MD has been received, the first data user U1 requests the first private data key P-DS1 from the manufacturer H in order to be able to decrypt the received machine data MD. The first data user U1 can now, in turn, encrypt the decrypted machine data MD with a public data key O-DS10 from its own stock of public data keys O-DS10, O-DS11 and transmit it to the second data user U2. When the second data user U2 has received the encrypted machine data MD, it requests the matching private data key P-DS10 from the manufacturer H and decrypts the received machine data MD with the matching private key P-DS10.

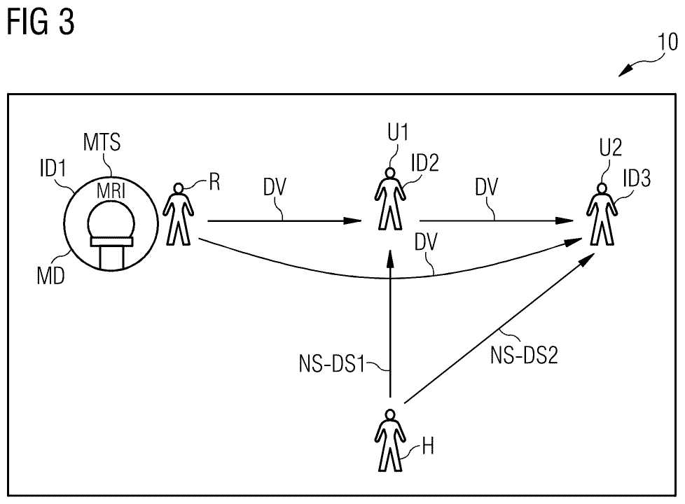

FIG. 3 is a schematic depiction of a data transfer system 10 according to yet another alternative exemplary aspect of the disclosure. Like the data transfer system 10 shown in FIG. 2, the data transfer system 10 shown in FIG. 3 is based on the concept that a participant in the data transfer system 10 is enabled to transmit encrypted data to the respective recipient even without prior authorization of the recipient by the manufacturer H. However, unlike the concept illustrated in FIG. 2, the concept illustrated in FIG. 3 does not require key pairs, each of which has a private data key and a public data key.

Instead, in the data transfer system 10 shown in FIG. 3, data to be transmitted, in particular machine data MD, is encrypted by an identification number ID1, ID2 of the data processing facility that transmits the data. For example, the data processing facility of the medical technology system MTS of the data transfer system 10 has an identification number ID1. If the data processing facility of the medical technology system MTS wishes to transmit the data to the first data user U1, said first data user can use its identification number ID1 to encrypt the data MD to be transmitted. The encrypted data DV can now be transmitted to any recipient. For example, the data processing facility of the medical technology system MTS transmits encrypted data DV to the first data user U1 of the data transfer system 10. The first data user U1 now requests a release key from the manufacturer H in the form of a user- and system-specific data key NS-DS1 for decrypting the data encrypted by the data processing facility of the medical technology system MTS. This user- and system-specific data key NS-DS1 alone allows the decryption of encrypted data DV of the data processing facility of the medical technology system MTS on a data processing facility of the first data user U1. For this purpose, the user- and system-specific data key NS-DS1 is based on an identification number ID2 of the data processing facility of the first data user U1 and the identification number ID1 of the data processing facility of the medical technology system MTS.

Therefore, the user- and system-specific data key NS-DS1 is restricted to one specific medical technology system MTS and one data user U1, so that no user has a “free pass” to use data from different medical technology systems. The encrypted data DV can also be transmitted with the identification number ID1 of the data processing facility of the medical technology system MTS either by the first data user U1 or by the data processing facility of the medical technology system MTS itself to a second data user U2.

In this case, the second data user U2 likewise asks the manufacturer H for a user- and system-specific data key and receives such a user- and system-specific data key NS-DS2, which is based on the identification number ID1 of the data processing facility of the medical technology system MTS and an identification number ID3 of the data processing facility of the second data user U2. The user- and system-specific decryption can, for example, take place on the basis of a manufacturer-specific secret sequence, which is used for decryption by a manufacturer-specific decryption program that is made available to the data users U1, U2. The aforementioned user- and system-specific data key NS-DS1, NS-DS2 is then a checksum for the identification numbers ID1, ID2 of the data processing facility of the medical technology system MTS and the data processing facility of the specific data user U1 and for the manufacturer-specific secret sequence, so that the manufacturer-specific decryption program only decrypts data from a specific medical technology system MTS for a specific data user U1. Therefore, unlike the exemplary aspects shown in FIG. 1 and FIG. 2, the exemplary aspect shown in FIG. 3 enables complete control of the system-data user relationship. In particular, a data user can only access the specific medical technology system MTS released for this data user in each case.

In the exemplary aspects illustrated in FIG. 1 and FIG. 2, it would, for example, be possible for the data users to exchange private data keys with one another and thus also for unauthorized users to access medical technology systems MTS. This is not possible in the aspect illustrated in FIG. 3, since this requires specific identification data for the parties involved in a transfer for decryption.

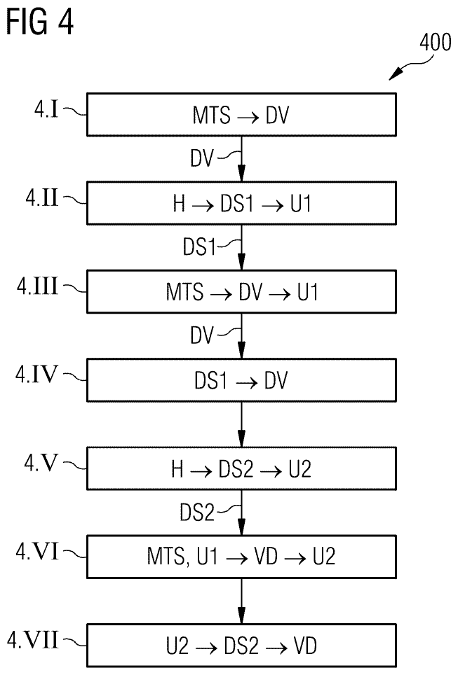

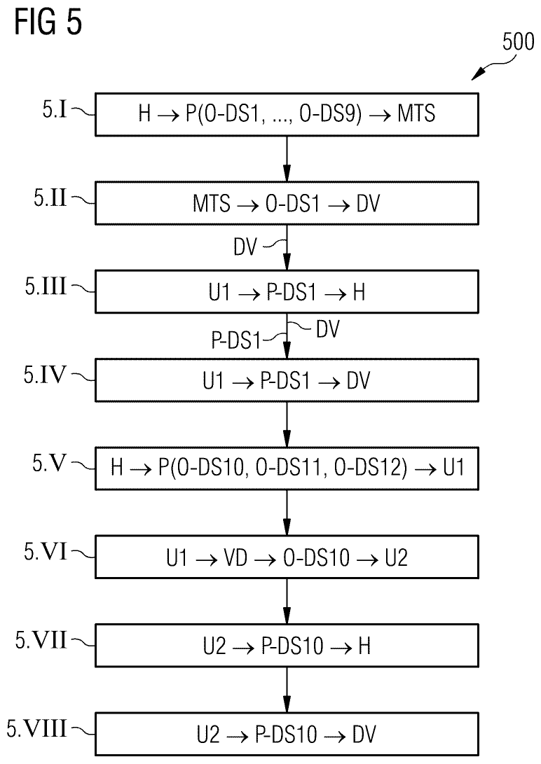

FIG. 4 shows a flowchart 400 illustrating a method for transmitting encrypted data DV from a medical technology system MTS according to an exemplary aspect of the disclosure.

In step 4.I, the encrypted data DV is generated by applying a data key DS0 specific to the medical technology system MTS to operating data of the medical technology system MTS.

In step 4.II, the first user-specific data key DS1 for decrypting the encrypted data DV is transmitted to a first data user U1 authorized by the central data transfer authorization entity H by the central data transfer authorization entity H.

In step 4.III, the encrypted data DV is transmitted from the medical technology system MTS to the first data user U1.

In step 4.IV, the encrypted data DV is decrypted by the first data user U1 by applying the first user-specific data key DS1.

In step 4.V, a second user-specific data key DS2, which is different from the first user-specific data key DS1, is transmitted by the central data transfer authorization entity H to a second data user U2 authorized by the central data transfer authorization entity H for decrypting the encrypted data DV.

In step 4.VI, the encrypted data DV is transmitted by the medical technology system MTS or by the first data user U1 to the second data user U2.

In step 4.VII, the encrypted data DV VD is decrypted by the second data user U2 by applying the second user-specific data key DS2.

FIG. 5 depicts a flowchart 500 illustrating a method for the monitored transmission of encrypted data DV from a medical technology system MTS according to an alternative exemplary aspect of the disclosure.

In step 5.I, the central data transfer authorization entity H transmits a pool P of public data keys O-DS1, . . . , O-DS9 to the medical technology system MTS.

In step 5.II, data, for example, machine data, is encrypted by the medical technology system MTS with one of the public data keys O-DS1 of the pool P before it is transmitted as encrypted data DV to the first data user U1.

In step 5.III, after receiving the encrypted data DV, the first data user U1 requests a first private key P-DS1 from the central data transfer authorization entity H.

In step 5.IV, the first data user U1 decrypts the encrypted data DV with the first private key P-DS1.

In step 5.V, the central data transfer authorization entity H transmits a pool of public second data keys O-DS10, O-DS11, O-DS12 to the first data user U1.

In step 5.VI, the first data user U1 encrypts the data decrypted in step 5.IV with a public data key O-DS10 of the received public second data keys O-DS10, O-DS11, O-DS12 and transmits this encrypted data DV to the second data user U2.

In step 5.VII, after receiving the encrypted data DV, the second data user U2 requests the second private key P-DS10 assigned to the public second data key DS10 used by the first data user U1 from the central data transfer authorization entity H.

In step 5.VII, after receiving the second private data key P-DS10, the second data user U2 decrypts the encrypted data DV with the second private data key P-DS10.

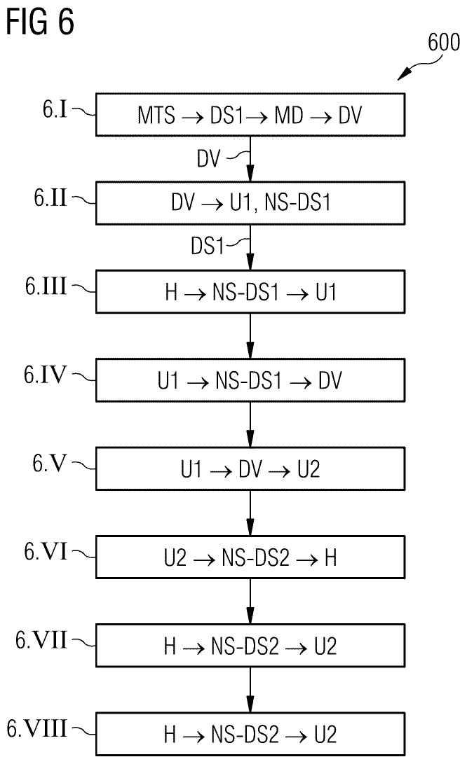

FIG. 6 shows a flowchart 600 illustrating a method for transmitting encrypted data DV from a medical technology system MTS according to yet another alternative exemplary aspect of the disclosure.

In step 6.I, the encrypted data DV is generated by applying a data key DS1 specific to the medical technology system MTS specific to machine data MD of the medical technology system MTS.

In step 6.II, a first data user U1 receives the encrypted data DV and asks the central data transfer authorization entity H for a user- and system-specific data key NS-DS1, which is configured specifically for the medical technology system MTS and the first data user U1.

In step 6.III, the desired user- and system-specific data key NS-DS1 is transmitted from the central data transfer authorization entity H to the first data user U1.

In step 6.IV, the encrypted data DV is decrypted by the first data user U1 by applying the first user-specific data key NS-DS1.

In step 6.V, the first data user U1 transmits the encrypted data DV to a second data user U1.

In step 6.VI, the second data user U2 asks the central data transfer authorization entity H for a user- and system-specific data key NS-DS2, which is specific to the medical technology system MTS and to the second data user U2.

In step 6.VII, the central data transfer authorization entity H transmits the desired user- and system-specific data key NS-DS2 to the second data user U2.

In step 6. VIII, the second data user U2 decrypts the encrypted data DV with the user- and system-specific data key NS-DS2 received.

Finally, reference is made once again to the fact that the above-described methods and apparatuses are merely preferred exemplary aspects of the disclosure and that the aspects of the disclosure can be varied by the person skilled in the art without leaving the scope of the disclosure as specified in the claims. For purposes of completeness, reference is also made to the fact that the use of the indefinite articles “a” or “an” does not preclude the possibility that the features in question may also be present on a multiple basis. Similarly, the term “unit” does not preclude the possibility that the unit may consist of a plurality of components that may also be spatially distributed. Independent of the grammatical term usage, individuals with male, female, or other gender identities are included within the term.

1. A method for transmitting encrypted data from a medical technology system, comprising:
generating the encrypted data by applying a data key to data generated by the medical technology system;
transmitting a first user-specific data key for decrypting the encrypted data to a first data user authorized by a central data transfer authorization entity to receive the encrypted data by the central data transfer authorization entity;
transmitting the encrypted data from the medical technology system to the first data user;
decrypting the encrypted data by the first data user by applying the first user-specific data key;
transmitting a second user-specific data key, which is different from the first user-specific data key, for decrypting the encrypted data from the central data transfer authorization entity to a second data user authorized by the central data transfer authorization entity to receive the encrypted data;
transmitting the encrypted data by the medical technology system or by the first data user to the second data user; and
decrypting the encrypted data by the second data user by applying the second user-specific data key.

2. The method as claimed in claim 1, wherein:
the second user-specific data key has a plurality of different data keys, and the second data user has a plurality of different data users and a different second user-specific data key, which is different from the first user-specific data key, and
the method further comprises:
transmitting the other second user-specific data keys to each of the different second data users by the central data transfer authorization entity,
transmitting the encrypted data by the medical technology system or by the first data user to the respective second data user, and
decrypting the encrypted data by the respective second data user by applying the respective second user-specific data key.

3. The method as claimed in claim 1, wherein the data generated by the medical technology system comprises a data type selected from the data types consisting of: machine data, measurement data from a region of interest, and medical image data.

4. The method as claimed in claim 1, wherein:
the first user-specific data key comprises a first data key pair with a first private data key and a first public data key,
the second user-specific data key comprises a second data key pair with a second private data key and a second public data key, and
the method further comprises:
transmitting the first public data key by the first data user to the medical technology system,
encrypting the data from the medical technology system with the first public data key before being transmitted to the first data user,
decrypting the encrypted data by the first data user after receipt with the first private data key,
transmitting the second public data key by the second data user to the medical technology system or to the first data user,
encrypting the encrypted data with the second public data key before transmitting to the second data user, and
decrypted the encrypted data by the second data user after receipt with the second private data key.

5. The method as claimed in claim 1, wherein the method further comprises:
transmitting a pool of public data keys by the central data transfer authorization entity to the medical technology system,
encrypting the data from the medical technology system by the medical technology system with one of the public data keys of the pool before transmitting to the first data user,
after receiving the encrypted data, requesting by the first data user a first private data key from the central data transfer authorization entity, and
decrypting the encrypted data with the first private data key.

6. The method as claimed in claim 5, wherein the method further comprises:
transmitting by the central data transfer authorization entity a pool of public second data keys to the first data user,
encrypting by the first data user the encrypted data with one of the public second data keys and transmitting it to the second data user,
after receiving the encrypted data, requesting by the second data user a second private data key assigned to the public second data key used by the first data user from the central data transfer authorization entity, and
after receipt of the second private data key, decrypting the encrypted data with the second private data key.

7. The method as claimed in claim 1, wherein:
the first user-specific data key comprises a first user- and system-specific data key for decrypting the data encrypted by applying a data key specific to the medical technology system, and
the second user-specific data key comprises a second user- and system-specific data key for decrypting the data encrypted by applying a data key specific to the medical technology system.

8. The method as claimed in claim 7, wherein the method further comprises:
generating the data key specific to the medical technology system based on a MAC address of a computer unit of the medical technology system,
generating the first user- and system-specific data key based on a MAC address of a computer unit of the first data user and based on the MAC address of the computer unit of the medical technology system, and
generating the second user- and system-specific data key based on a MAC address of a computer unit of the second data user and based on the MAC address of the computer unit of the medical technology system.

9. The method as claimed in claim 8, wherein the method further comprises:
generating the data key specific to the medical technology system based on a secret code generated by the data transfer authorization entity,
generating the first user- and system-specific data key based on the secret code generated by the data transfer authorization entity, and
generating the second user- and system-specific data key based on the secret code generated by the data transfer authorization entity.

10. The method as claimed in claim 9, wherein the method further comprises:
generating the first user- and system-specific data key based on a checksum for the secret code generated by the data transfer authorization entity, the MAC address of the computer unit of the medical technology system, and the MAC address of the computer unit of the first data user, and
generating the second user- and system-specific data key based on a checksum for the secret code generated by the data transfer authorization entity, the MAC address of the computer unit of the medical technology system, and the MAC address of the computer unit of the second data user.

11. The method as claimed in claim 1, wherein the first and/or second user-specific data key or the first and/or second user- and system-specific data key has a limited validity period.

12. The method as claimed in claim 1, wherein the first and/or second user-specific data key or the first and/or second user- and system-specific data key are assigned to a specific authorization level.

13. A data transfer system, comprising:
a medical technology system that is configured to generate encrypted data by applying a data key specific to the medical technology system to operating data of the medical technology system;
a first data user;
a central data transfer authorization entity that is configured to authorize the first data user to receive the encrypted data and to transmit a first user-specific data key for decrypting the encrypted data to the first data user authorized by the central data transfer authorization entity,
wherein the medical technology system is configured to transmit the encrypted data to the first data user, and
wherein the first data user is configured to decrypt the encrypted data by applying the first user-specific data key; and
a second data user,
wherein the central data transfer authorization entity is configured to authorize the second data user to receive the encrypted data and to transmit a second user-specific data key, which is different from the first user-specific data key, for decrypting the encrypted data to the second data user authorized by the central data transfer authorization entity,
wherein the medical technology system and/or the first data user are configured to transmit the encrypted data to the second data user, and
wherein the second data user is configured to decrypt the encrypted data by applying the second user-specific data key.

14. A non-transitory computer-readable medium comprising instructions, which, when executed by a computer, cause the computer to execute the steps of the method as claimed in claim 1.
